(19) United States
(12) Patent Application Publication
(10) Pub. No.: US 2022/0062160 A1
(43) Pub. Date: Mar. 3, 2022

(54) METHOD FOR INHIBITING SKIN ACNE FORMATION, REDUCING CUTIBACTERIUM ACNES SECRETIONS, AND/OR REDUCING ADVANCED GLYCATION END-PRODUCTS USING WASABIA JAPONICA LEAF EXTRACT

(71) Applicant: TCI CO., LTD., Taipei (TW)

(72) Inventors: YUNG-HSIANG LIN, Taipei (TW); TSAI-HAN YAO, Taipei (TW)

(21) Appl. No.: 17/513,916

(22) Filed: Oct. 29, 2021

(30) Foreign Application Priority Data
Sep. 2, 2020 (TW) 109130113

(51) Int. Cl. A61K 8/9789; A23L 33/105; A61Q 19/08

(52) U.S. Cl. CPC A61K 8/9789; A61K 2800/805; A61Q 19/08; A23L 33/105

(57) ABSTRACT

A method for inhibiting skin acne formation, reducing Cutibacterium acnes secretions, and/or reducing advanced glycation end-products (AGEs) comprising administering to a subject in need thereof a composition comprising an effective amount of a Wasabia japonica leaf extract is provided. The composition is used for increasing skin water content, improving skin firmness, reducing skin wrinkles, inhibiting the production of skin wrinkles, inhibiting the production of skin spots, inhibiting the production of melanin, and inhibiting the production of acne.

### CROSS-REFERENCE TO RELATED APPLICATION

This non-provisional application claims priority under 35 U.S.C. § 119(a) to Patent Application No. 109130113 filed in Taiwan, R.O.C. on Sep. 2, 2020, the entire contents of which are hereby incorporated by reference.

### REFERENCE OF AN ELECTRONIC SEQENCE LISTING

The contents of the electronic sequence listing (P201069USI_ST25_F.txt; Size: 0.6 KB; and Date of Creation: Oct. 29, 2021) is herein incorporated by reference in its entirety.

### BACKGROUND

### Technical Field

The present disclosure relates to a method for delaying skin aging or changing skin condition by using a combination, and in particular, to a combination comprising an effective amount of a Wasabia japonica leaf extract.

### Related Art

Skin is of indispensable importance for protection of human individuals, it provides first-stage protection against environments such as ultraviolet radiation in the sun, pathogens, and friction. Skin sequentially includes, from outside to inside, the epidermis layer, the dermis layer mainly formed by the connective tissue, and the subcutaneous tissue. The dermis layer contains :sufficient molecules such as collagen, elastin, and hyaluronic acid, so that young skin is more supportive and has a better elasticity.

With aging or the influence of environments, skin will age and bear various damage conditions. For example, the skin may have a decreased elasticity, an insufficient water retention (which means that the skin is dry and dull), enlarged pores, wrinkles, etc. caused by collagenolysis. In another example, the skin has spots and/or the overall skin is dull, sallow, or in other aging conditions due to melanotic hyperpigmentation.

In addition, substances that accelerate aging—advanced glycation end-products (AGEs) may be formed by the combination of excess sugar and protein in the blood. The accumulation of these AGEs in the body will also cause the skin to appear aging and damaged. Especially in modem life, people often have excessive sugar intake with other factors such as lack of exercise or slow metabolism, and thus causing the skin aging more obvious and rapid.

### SUMMARY

In view of this, it is necessary to research or develop a composition that can delay skin aging and reduce skin damage to maintain or improve the skin appearance.

Therefore, an objective of the present disclosure is to provide a method for delaying skin aging or changing skin condition by using a composition comprising an effective amount of a Wasabia japonica leaf extract. Wasabia japonica, also referred to as Eutrema japonicum, is a plant of the family Brassicaceae. Wasabia japonica has a strong pungent taste that is different from the spicy taste of chili. The spicy taste of chili stimulates the tongue, while the pungent taste of Wasabia japonica stimulates the nose. Wasabia japonica leaves are edible and have the pungent taste of Wasabia japonica stems. Ways of cooking Wasabia japonica leaves include the following: Wasabia japonica leaves are marinated in salt and vinegar sauce overnight for a Wasabia japonica salad; Wasabia japonica leaves are boiled in soybean sauce and boiled water as a side dishes that tasted well with rice and wine or an ingredient for sushi rolls; and Wasabia japonica leaves can even be dipped in a starch paste and then fried into tempura.

Specifically, an objective of the present disclosure is to provide a method for inhibiting skin acne formation, reducing Cutibaceterium acnes secretions, and/or inhibiting the production of advanced glycation end-products (AGEs), increasing skin water content, improving skin firmness, reducing skin wrinkles, inhibiting the production of skin wrinkles, inhibiting the production of skin spots, inhibiting the production of melanin, and inhibiting the production of acne by using a composition comprising an effective amount of a Wasabia japonica leaf extract.

In some embodiments, the composition is a pharmaceutical composition.

In some embodiments, the composition is a cosmeceutical composition, a health food composition, or a cosmetic composition.

In some embodiments, the composition is used for inhibiting the production of skin acne.

In some embodiments, the Wasabia japonica leaf extract is extracted from a Wasabia japonica leaf with a water-containing solvent.

In some embodiments, the Wasabia japonica leaf extract is used for delaying skin aging by reducing the production of AGEs.

In some embodiments, the Wasabia japonica leaf extract is used for inhibiting the production of skin acne by regulating the gene expression of anti-inflammatory genes.

In some embodiments, the concentration of the Wasabia japonica leaf extract is at least 0.5 mg/ml.

In some embodiments, the Wasabia japonica leaf extract can reduce the amount of water lost through skin, increase skin water content, improve skin elasticity, improve skin firmness, fade skin spots, fade skin UV spots, improve skin texture, improve skin pore condition, reduce skin wrinkles, reduce skin porphyrin, or resolve any combination of the foregoing problems.

As described above, excess sugar and protein in the blood will be combined to form AGEs. The continuous accumulation of AGEs will cause inflammation, and then generate free radicals to attack skin, resulting in skin-related protein hardening and fiber breakage, so that the skin will lose its elasticity, and become dull, sallow, wrinkled, rough, and severely sagging, and thus accelerates skin aging.

In addition, when the oil and old wasted cells on the skin accumulate, bacteria will breed, causing slight skin inflammation. If the inflammation is not treated in time, the hair follicle wall of the skin will be damaged, causing a severe inflammation and the production of acne (or so-called “pimple”). A very important part of the inflammation is a proinflammatory cytokine. If the secretion of the proinflammatory cytokine can be reduced, the inflammation can be reduced to achieve the effect of anti-acne.

In another aspect, ultraviolet light and blue light can also induce melanocytes in the skin to produce melanin, causing the skin to produce spots and/or the overall skin to be dull and sallow. This is also one of the common phenomena of skin aging.

In addition, because it is confirmed in the present disclosure that the Wasabia japonica leaf extract can inhibit the production of AGEs, inhibit the proinflammatory cytokines, and in particular, can inhibit the proinflammatory cytokines and inhibit the production of melanin by regulating the gene expression of IL-8, the Wasabia japonica leaf extract of the present disclosure can effectively delay skin aging and inhibit the production of skin acne, and can also achieve whitening and against various skin aging phenomena.

### BRIEF DESCRIPTION OF THE DRAWINGS

In the figures, “*” represents a p-value less than 0.05, “**” represents a p-value less than 0.01, and “***” represents a p-value less than 0.001. More “*” represents more significant statistical differences.

FIG. 1 is a result comparison diagram of the relative production of AGEs between a blank control group and an experimental group;

FIG. 2 is a result comparison diagram of the relative gene expression ratio of IL-8 between a blank control group and an experimental group;

FIG. 3 is a result comparison diagram of the relative secretion ratio of IL-8 among a blank control group, an ultra violet B (UVB) control group, and an experimental group;

FIG. 4 is a result comparison diagram of the relative content ratio of melanin among a blank control group, a control group, and an experimental group;

FIG. 5 is a result comparison diagram of the relative inhibition of tyrosinase between a blank control group and an experimental group; and

FIG. 6 is a comparison diagram of the improvement ratio of the skin condition (the transepidermal water loss, skin moisture, skin elasticity, skin firmness, spots, UV spots, skin texture, skin pores, skin wrinkles, porphyrin (Cutibacterium acnes secretion)) of subjects before and after drinking a drink containing the Wasabia japonica leaf extract of the present disclosure.

### DETAILED DESCRIPTION

The following will describe some specific implementations of the present disclosure. Without departing from the spirit of the present disclosure, the present disclosure can still be practiced in many different forms, and the protection scope should not be limited to the conditions specified in this specification.

In the present disclosure, statistical analysis is conducted by using Excel software, Data is expressed as mean±standard deviation (SD), and the differences between groups are analyzed by student's t-test.

Numerical values used herein are approximate values, and all experimental data are expressed within the range of ±10%, and the best within the range of ±5%.

In the present disclosure, Wasabia japonica, also referred to as Eutrema japonicum, is a plant of the family Brassicaceae.

In some embodiments, the Wasabia japonica leaf extract may be used for delaying skin aging, inhibiting the production of skin acne, increasing skin water content, improving skin firmness, reducing skin wrinkles, inhibiting the production of skin wrinkles, inhibiting the production of skin spots, and inhibiting the production of skin melanin. Therefore, the Wasabia japonica leaf extract may be used to prepare a composition for delaying skin aging, inhibiting the production of skin acne, increasing skin water content, improving skin firmness, reducing skin wrinkles, inhibiting, the production of skin wrinkles, inhibiting the production of skin spots, and inhibiting the production of skin melanin.

In some embodiments, the Wasabia japonica leaf extract may inhibit the production of tyrosinase by reducing the production of AGEs, regulating the gene expression of anti-inflammatory genes, regulating the gene expression of IL-8, and regulating the gene expression of MTF.

In some embodiments, the composition may be a pharmaceutical composition, a cosmeceutical composition, a food composition, or a health food composition.

The pharmaceutical composition may be manufactured into a dosage form suitable for enteral, parenteral, or topical administration by using techniques well known to those skilled in the art. For example, the pharmaceutical composition may be an injection (such as a sterile aqueous solution or a dispersion), a sterile powder, an external preparation, or other similar substances.

The pharmaceutical composition may further comprise a pharmaceutically acceptable carrier widely used in drug manufacturing technology. For example, the pharmaceutically acceptable carrier may comprise one or more of the following carriers: an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and other similar substances. The related selection and quantity of these carriers may be selected by those skilled in the art according to the situation.

In some embodiments, the pharmaceutically acceptable carrier may include the following solvents: water, normal saline, phosphate buffered saline (PBS), aqueous solution containing alcohol, and any other proper solvents.

In some embodiments, the pharmaceutical composition may be administered by any one of the following parenteral routes: subcutaneous injection, intraepidermal injection, intradermal injection, and intralesional injection.

In some embodiments, the pharmaceutical composition may be manufactured into an external preparation suitable for topical application to skin by using techniques well known to those skilled in the art. For example, the pharmaceutical composition may be any one of the following., but not limited to: an emulsion, a gel, an ointment, a cream, a patch, a liniment, a powder, an aerosol, a spray, a lotion, a serum, a paste, a foam, a drop, a suspension, a salve, and a bandage.

In some embodiments, the external preparation is made by mixing the pharmaceutical composition with a base well known to those skilled in the art.

In some embodiments, the base may comprise one of more of the following additives: water, alcohols, glycol, hydrocarbons (such as petroleum jelly and white petrolatum), wax (such as paraffin and yellow wax), preserving agents, antioxidants, surfactants, absorption enhancers, stabilizing agents, gelling agents (such as carbopol® 974P, microcrystalline cellulose, and carboxymethyl cellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants, etc. The related selection and quantity of these additives are falling within the scope of professiomilism and routine technology of those skilled in the art.

In some embodiments, the cosmeceutical may comprise an acceptable adjuvant widely used in cosmeceutical manufacturing technology. For example, the acceptable adjuvant may comprise one or more of the following adjuvants: a solvent, a gelling agent, an active agent, a preservative, an antioxidant, a screening agent, a chelating agent, a surfactant, a coloring agent, a thickening agent, a filler, a fragrance, and an odor absorber. The related selection and quantity of these adjuvants may be properly adjusted according to actual needs.

In some embodiments, the cosmeceutical may be made into a form suitable for skincare or makeup by using techniques well known to those skilled in the art. The cosmeceutical may be any one of the following, but not limited to: an aqueous solution, an aqueous-alcohol solution or an oily solution, and an emulsion, a gel, an ointment, a cream, a mask, a patch, a pack, a liniment, a powder, an aerosol, a spray, a lotion, a serum, a paste, a foam, a dispersion, a drop, a mousse, a sunblock, a tonic water, a foundation, a makeup remover product, a soap, and other body cleansing products that are an oil-in-water type, a water-in-oil type, or a composite type.

In some embodiments, the cosmeceutical may also be used in combination with one or more of the following external use agents with known activity: whitening agents (such as tretinoin, catechin, kojic acid, arbutin, and vitamin C), humectants, bactericides, ultraviolet absorbers, plain extracts (such as aloe extracts), skin nutrients, anesthetics, anti-acne agents, antipruritics, analgesics, antidermatitis agents, antihyperkeratolytic agents, anti-dry skin agents, antipsoriatic agents, antiaging agents, antiwrinkle agents, antiseborrheic agents, wound-healing agents, corticosteroids, and hormones. The related selection and quantity of these external use agents are falling within the scope of professionalism and routine technology of those skilled in the art.

In some embodiments, the pharmaceutical composition may be used as a food additive. It is added during the preparation of raw materials or added during the food making by conventional methods to form a food product for humans and non-human animals to eat with any edible material.

In some embodiments, the type of food may be, but is not limited to: beverages, fermented foods, bakery products, health foods, and dietary supplements.

In some embodiments, the Wasabia japonica leaf extract may be extracted from Wasabia japonica leaves with a solvent, and the solvent may be water or a water-containing solution. In some embodiments, the Wasabia japonica leaf extract is extracted at 70-100° C., and the extraction time is 30 min. In some embodiments, the Wasabia japonica leaf extract is obtained by performing extraction on Wasabia japonica leaves with a proper solvent, squeezing the Wasabia japonica leaves and removing leaf residues and fine suspended substances, and then concentrating it to a certain concentration.

In some embodiments, the Wasabia japonica leaf extract may alternatively be obtained by directly crushing and squeezing Wasabia japonica leaves and removing leaf residues and fine suspended substances, and then concentrating it to a certain concentration.

Unless otherwise stated, the experimental steps in the following examples are carried out at room temperature (25±5° C.) and normal pressure (1 atm).

### EXAMPLE 1: Preparation of Wasabia japonica Leaf Extract

Wasabia japonica leaves (origin: Alishan, Taiwan and Dayi County, Chengdu) were crushed (by a 10-speed blender of brand Osterizer) and sieved with a 10-mesh sieve to remove large particles to obtain a Wasabia japonica leaf powder. In some other embodiments, the mesh of the sieve may be 8 meshes or 12 meshes.

Next, in the heating process, water as a solvent and the Wasabia japonica leaf powder were mixed in a weight ratio of 15:1, and extraction was carried out at 85±5° C. for about 30 min, to form a first extracted liquid containing solids.

In some other embodiments, the weight ratio of the solvent to the Wasabia japonica leaf powder may be 10:1-20:1, the soaking temperature may be 70-100° C., and the extraction time may be 15-45 min. If the amount of the solvent is too little or the extraction time is too short, the extraction efficiency will be significantly reduced. If the extraction time is too long, the active ingredients in the extract may be degraded.

The foregoing first extracted liquid was concentrated under reduced pressure at 60±5° C. by using a concentrator (brand/model: BUCHI-Rotavapor R-100) until the Brix degree of the solution was 8±0.5°, to obtain the Wasabia japonica leaf extract of the present disclosure. In some other embodiments, the concentration under reduced pressure may be carried out at 45-70° C.

### EXAMPLE 2: Detection of the Amount of Collagen Glycation Inhibited by Wasabia japonica Leaf Extract

Various AGEs are produced after protein undergoes glycation, which are non-reversible substances and can change and affect normal functions of the protein, thereby accelerating skin aging. Therefore, if collagen glycation can be inhibited, the loss rate of the collagen can be effectively reduced, thereby achieving the effect of delaying skin aging. However, to confirm whether the Wasabia japonica leaf extract has the effect of inhibiting collagen glycation, the amount of collagen glycation is tested as follows.

Drug Preparation:

The Wasabia japonica leaf extract prepared as in Example 1 and 200 mM phosphate-buffered saline (PBS, prepared by NaH₂PO₃ (Honeywell, #04269), Na₂HPO₄ (Sigma, #V900061), and water, with a pH value of 7.4) were used to prepare a Wasabia japonica leaf extraction solution with a concentration of 0.4 mg/mL.

The 200 mM PBS and a collagen powder (the collagen with a model of #P2000HD was purchased from Rousselot) were used to prepare a collagen solution with a concentration of 60 mg/mL. In addition., sodium azide was added into the collagen solution to allow the collagen solution to contain 0.06 wt % of sodium azide. (The sodium azide with a model of #S2002 was purchased from Sigma.)

The 200 mM PBS and fructose (the fructose with a model of #F0127 was purchased from Sigma) were used to prepare a fructose solution with a concentration of 1.5 M.

Test of Collagen Glycation:.

0.2 mL of the Wasabia japonica leaf extraction solution was mixed with 0.2 mL of the collagen solution and 0.2 mL of the fructose solution to prepare a sample solution.

0.2 mL of deionized water was mixed with 0.2 mL of the collagen solution and 0.2 mL of the fructose solution to prepare a blank solution as a control group.

Before collagen glycation was carried out, the fluorescence intensities of the sample solution and the blank solution were measured by using a spectrofluorometer (brand/model: BioTek FLx 800, with an excitation wavelength of 360 nm and an emission wavelength of 460 nm).

The sample solution and the blank solution were reacted at 50° C. for 24 h to undergo the collagen glycation.

The fluorescence intensities of the sample solution and the blank solution after reaction were also measured by using a spectrofluorometer (with an excitation wavelength of 360 nm and an emission wavelength of 460 nm).

The relative production of protein AGEs was calculated according to the following formula.:

[(sample fluorescence intensity_{after reaction}−sample fluorescence intensity_{before reaction})/(control fluorescence intensity_{after reaction}−control fluorescence intensity_{before reaction})]×100%

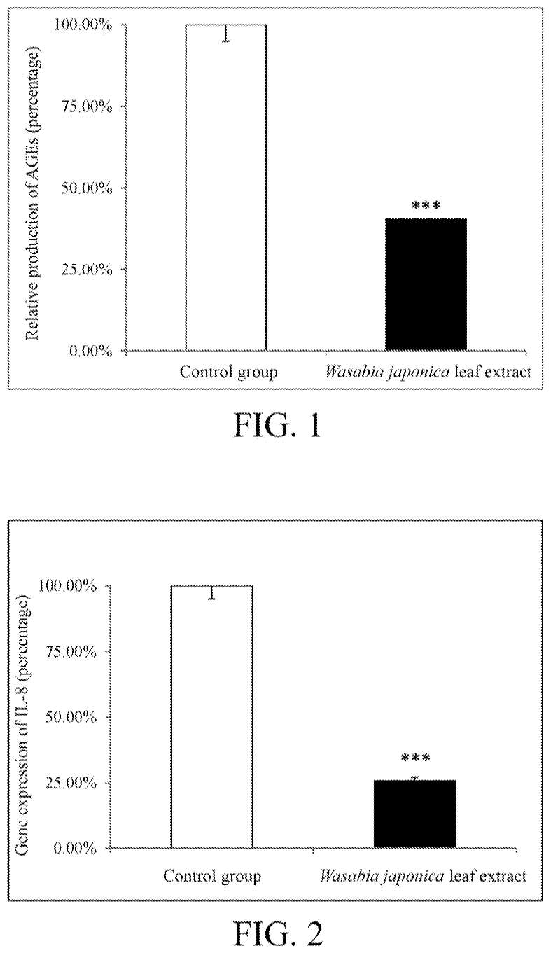

The relative production of protein AGEs of the sample relative to the control group is shown in FIG. 1. As shown in FIG. 1, the Wasabia japonica leaf extract has the effect of inhibiting the production of collagen AGEs. Specifically, the relative production of protein AGEs of the sample is only 41% of that of the control group. It indicates that the Wasabia japonica leaf extract can indeed inhibit the collagen glycation, reduce the production of AGEs, and further reduce the loss of collagen in the body, thereby achieving the effect of delaying skin aging.

### EXAMPLE 3: Cell Experiment—Regulation of the Gene Expression of IL-8 by Wasabia japonica Leaf Extract

A culture medium used was VIVOTM 10 Serum-free hematopoietic cell medium (purchased from Lonza, Switzerland, No. BE02-055Q). First, the experiment of the gene expression of IL-8 of vascular endothelial cells was carried out on human umbilical vein endothelial cells (HUVECs, obtained from the Bioresource Collection and Research Center (BCRC), No. H-UV001). The cells were first cultured in a six-well culture plate containing 2 mL of the culture medium in a density of 1×10⁵ cells per well at 37° C. for 16 h, and the HUVECs were then divided into the following two groups: an experimental group and a control group.

The experimental group: an extract-containing culture medium was prepared according to a ratio of 1 mg of the Wasabia japonica leaf extract prepared as in Example 1 to 1 mL of the culture medium (that is, the concentration is 1 mg/mL), the medium was changed to the extract-containing culture medium, and the HUVECs were further cultured therein.

The control group: no treatment is performed, that is, no additional compounds were added into the culture medium containing the cultured HUVECs.

After the experimental group and the control group were cultured for 48 h, the cell membranes of the cultured cells in the experimental group and the control group were broken with a cell lysis buffer to form two groups of cell solutions.

Next, RNA of the two groups of cell solutions was collected separately by using an RNA extraction reagent kit (purchased from Geneaid, Taiwan, Lot No. FC24015-G). Then, 2000 ng of the extracted RNA in each group was used as a template, and reverse transcription was carried out b anealing in Table 1 through the SuperScript® III reverse transcriptase (purchased from Invitrogene, USA, No. 18080-051) to generate corresponding cDNA. Subsequently, the quantitative real-time reverse transcription polymerase chain reaction was carried out on the two groups of reverse transcribed products respectively with the annealing in Table 1 by using the ABI StepOnePlus™ Real-Time PCR system (Thermo Fisher Scientific., USA) and the KAPA SYBR FAST (purchased from Sigma, USA, NO. 38220000000) to observe the gene expression of HUVEC of the experimental group and the control group. The instrument setting conditions for the quantitative real-time reverse transcription polymerase chain reaction were 95° C. for 1 s, 60° C. for 20 s, a total of 40 cycles, and gene quantification by the 2-ΔCt method. Therefore, the quantitative real-time reverse transcription polymerase chain reaction with cDNA can indirectly quantify the mRNA expression of each gene, and then infer the expression of the protein encoded by each gene.

TABLE 1

| Primer | | | |
|---|---|---|---|
| name | Sequence NO. | Sequence | Length |
| IL-8-F | SEQ ID NO: 1 | TTTTGCCAAGGAGTGCTAAAGA | 22 |
| IL-8-R | SEQ ID NO: 2 | AACCCTCTGCACCCAGTTTTC | 21 |

The relative gene expression of each gene shown in the drawings mentioned below is presented in relative scale. The standard deviation is calculated by using the STDEV formula of Excel software, and whether there is a statistically significant difference is analyzed by one-tailed student's t-test in Excel software. In the figures, “*” represents a p-value less than 0.05, “**” represents a p-value less than 00.1, and “***” represents a. p-value less than 0.001. More “*” represents more significant statistical differences.

As shown in FIG. 2, when the gene expression of IL-8 in the control group is regarded as 1 (i.e., 100%), the gene expression of IL-8 in the experimental group relative to the control group is 0.258 (i.e., 25.8%), indicating that the gene expression of IL-8 in the experimental group is 0.258 times that in the control group.

It can be learned that the gene expression of 1L-8 in HUVECs after the HUVECs are treated with the Wasabia japonica leaf extract, indicating that the inflammation in cells can be effectively inhibited, and therefore the production of acne can be effectively inhibited, thereby achieving the effect of anti-acne.

### EXAMPLE 4: Cell Experiment—Evaluation of the Effectiveness of Wasabia japonica Leaf Extract in Anti-Skin Inflammation

It is known that lL-8 is a cytokine that has the function of promoting inflammation. When inflammation occurs, the inflamed tissue will release the cytokine to attract cells with specific functions to the inflammation sites. In addition, when inflammation occurs, the sites will be red and swollen, and even painful. Therefore, if the secretion of IL-8 can be inhibited, the inflammation can be alleviated, thereby achieving the effect of inhibiting skin acne.

In view of this, the changes of anti-inflammatory factors in human primary epidermal keratinocytes HPEK-50 after treated by the Wasabia japonica leaf extract are measured by using a human CXCL8/IL-8 ELISA analysis kit and an ultraviolet irradiation room with an ELISA reader.

Material and Instrument:
1. Cell strain: human primary epidermal keratinocytes HPEKp (CELLnTEC, Switzerland, HPEK-50).
2. Culture medium: keratinocyte-serum-free medium (keratinocyte-SFM, Gibco, USA, No. #10724-011).
3. Ultraviolet irradiation room (Vilber, No. BLX254).
4. Human CXCL8/IL8 ELISA analysis kit: purchased from R&D systems, model D8000CD8000C, including the following reagents:
(1) capture antibody
(2) detection antibody
(3) streptavidin-HRP
(4) washing buffer: PBS solution
(5) tetramethyl-benzidine (TMB) substrate solution
(6) stop solution
ELISA reader, purchased from BioTek.
6. substrate solution (R&D systems).
7. H₂SO₄, purchased from Sigma-Aldrich.
8. cell incubator, purchased from Astec.
9. shaker, purchased from GenePure.
10. Wasabia japonica leaf extract: the Wasabia japonica leaf extract used in this experiment is prepared according to Example 1.

Experimental Steps:

The experiment is divided into three groups: a control group, a UVB irradiation group, and an experimental group, and the experiment in each group is repeated for three times.
1. HPEKs were inoculated in a 24-well culture plate containing 0.5 mL of culture medium per well in a density of 5×1.0⁴ cells per well.
2. The culture plate was placed in a cell incubator for culture at 37° C. for 24 h, and then the culture medium was removed.
3. Inflammation was induced in the UVB irradiation group and the experimental group at. an intensity of 300 mJ/cm² by using an ultraviolet irradiation room, and 1 mg/mL of Wasabia japonica leaf extract sample was added into cells of the experimental group. No treatment was performed on cells of the control group (that is, no UVB irradiation and no addition of the Wasabia japonica leaf extract). The Wasabia japonica leaf extract sample was a solution containing 1 mg of the Wasabia japonica leaf extract per mL prepared by diluting the Wasabia japonica leaf extract prepared as in Example 1 with a culture medium.
4. After each group of cells were cultured at 37° C. for 24 h, 120 μL of cell culture supernatant was taken as a sample, and IL-8 in these samples was analyzed by using a human CXCL8/IL8 ELISA analysis kit.
5. First, the capture antibody was diluted with PBS, and the diluted capture antibody was applied to the bottom of a 96-well microplate in a density of 100 μL per well and reacted overnight at 4° C.
6. Next, the 96-well microplate was washed for three times with 200 μL of washing buffer (containing 0.05% of Tween 20, and prepared in PBS).
7. Blocking was carried out by using 300 μL of block buffer (containing 1% of BSA, and prepared in PBS) to sequentially react at 37° C. for 3 h.
8. Then, the 96-well microplate was washed for three times with 200 μL of washing buffer.
9. 100 μL of sample and standard (prepared in a diluent (containing 0.1% of BSA, and prepared in PBS)) were added and then bound to the capture antibody at 37° C. for 2 h.
10. Then, the 96-well microplate was washed for three times with 200 μL of washing buffer.
11. 100 μL of detection antibody was then added to detect the capture antibody at 37° C. for 2 h.
12. Then, the 96-well microplate was washed for three times with 200 μL of washing buffer.
13. 100 μL of streptavidin-HRP was added to react at room temperature for 20 min.
14. Then, the 96-well microplate was washed for three times with 200 μL of washing buffer.
15. 100 μL of substrate solution (R&D systems) was then added to react at room temperature for 20 min.
16. Then, 50 μL of stop solution was added to terminate the reaction. Finally, the absorbance at 450 nm was measured by an ELISA reader. Statistical analysis was then conducted by using Excel software.

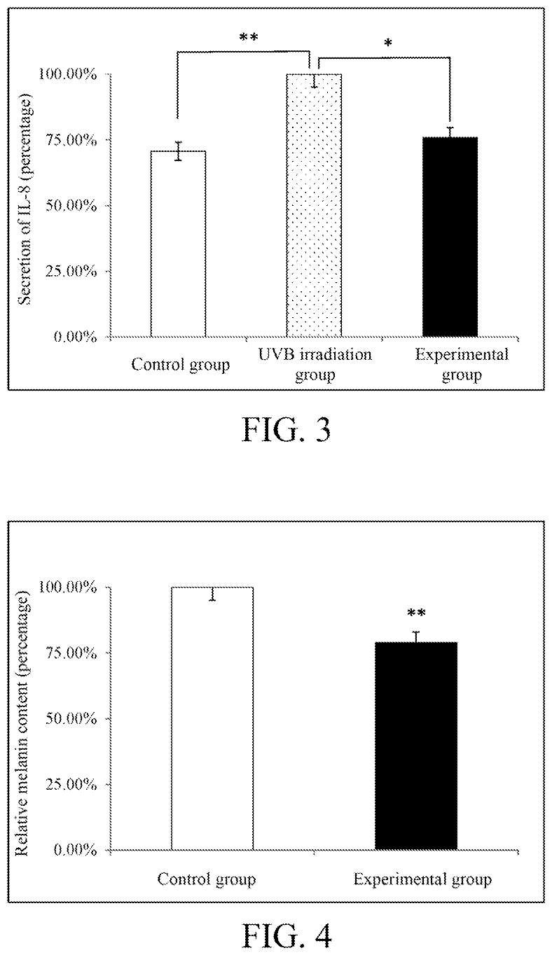

The statistically significant differences between groups were determined by student's t-test. The result of this Example is shown in FIG. 3.

FIG. 3 is a data diagram of the effect of the Wasabia japonica leaf extract of the present disclosure in anti-skin inflammation. As shown in FIG. 3, compared with the control group, the production of IL-8 in the UVB group has a significant increase, indicating that HPEKs have an inflammation with UVB. Compared with the UVB group, the production of IL-8 in the experimental group is reduced by 14.3%. The result of this Example shows that the Wasabia japonica leaf extract of the present disclosure has the effect of anti-skin inflammation, thereby achieving the effect of inhibiting the production of skin acne and anti-acne.

### EXAMPLE 5: Cell Experiment—Reduction of the Production of Melanin by Wasabia japonica Leaf Extract

The melanoma cell strain B16F10 was treated with the Wasabia japonica leaf extract and then measured by an ELISA reader for the changes in the content of melanin.

Material and Instrument:
1. Cell strain: mouse melanoma cell strain B16F10 (ATCC CRL-6475), purchased from the American Type Culture Collection (ATCC).
2. Culture medium: additional ingredients were added to the Dulbecco's modified minimal essential medium (DMEM, purchased from Gibco) to allow the medium to contain 1 vol % of antibiotic antimycotic solution (purchased from Gibco, 15240-06.2) and 10 vol % of fetal bovine serum (FBS, purchased from Gibco, 10437-028).
3. PBS solution: purchased from Gibco, product No. 10437-028).
4. 1 N NaOH (purchased from Sigma, product No. 221465) solution was prepared with double-distilled water.
5. ELISA reader (BioTek, FLx 800).
6. Wasabia japonica leaf extract prepared as in Example 1.

Experimental Steps:

The experiment is divided into two groups: an experimental group and a blank control group (no addition of the Wasabia japonica leaf extract), and the experiment in each group is repeated for three times.
1. B16F10 cells were inoculated in a 6-well culture plate containing 2 mL of culture medium per well in a density of 1.5×10³ cells per well.
2. The culture plate was placed in an environment of 5% CO₂ and 37° C. to carry out culture for 24 h.
3. Then, the culture medium per well was removed without disturbing attached cells.
4. The experimental group: 0.0225 of the Wasabia japonica leaf extract sample was added into each well for culture at 37° C. for 48 h. The Wasabia japonica leaf extract sample was a solution containing 1 mg of the Wsabia japonica leaf extract per milliliter prepared by diluting the Wasabia japonica leaf extract prepared as in Example 1 with a culture medium.
The blank control group: 2 mL of culture medium was added into each well at 37° C. for culture at 37° C. for 48 h.
The control group: 2 mL of culture medium was added into each well at 37° C. for culture at 37° C. for 48 h.
5. Then, the culture medium was removed, and the cells were washed twice with the PBS solution.
6. 200 μL of trypsin-EDTA (10×) (purchased from Gibco; product No. 15400-054) was added into each well to react for 3 min. After the reaction, 6 m L of the culture medium solution was added to terminate the reaction. Then, the suspension cells and culture medium in each well were collected into a 15 mL centrifuge tube, and each centrifuge tube was centrifuged at 400 × g for 5 min to pellet the cells.
7. After the pelleted cells were washed twice with the PBS solution, cells were resuspended with 200 μL of PBS solution.
8. The cell suspension was frozen in liquid nitrogen for 10 min and then placed at room temperature for about 30 min until completely unfrozen.
9. The tube was centrifuged at 12,000 g for 3 min after completely unfrozen.
10. The supernatant was removed, the pelleted cells were resuspended with 120 μL of 1 N NaOH solution, and then the tube was placed in a dry bath at 60° C. for 1 h, to obtain a to-be-detected sample.
11. 100 ↑L of the to-be-detected sample was transferred into a 96-well plate, and the optical density OD₄₅₀ of the cell solution at 450 nm was measured by the ELISA reader as the absorbance.

Experimental Result:

The relative expression of melanin of the experimental group, the blank control group, and the control group were calculated according to the following formula: relative expression of melanin (%)=(OD₄₅₀ value of each group/OD₄₅₀ value of blank control group)×100%. The experiment was repeated for three times, so that the results of the three repeated experiments were averaged and then shown in FIG. 4.

As shown in FIG. 4, it can be learned from the results of the blank control group and the control group that the expression of melanin in cells increases significantly after blue-light irradiation, indicating that the blue-light irradiation can indeed cause melanoma cells to produce melanin, which will cause the skin to produce spots or the overall skin to be dull. According to another aspect, it can be learned according to the results of the control group and the experimental group that the expression of melanin of the cells treated with the Wasabia japonica leaf extract under blue-light irradiation is lower than that of the control group that is not treated with the Wasabia japonica leaf extract (a decrease of about 21%), indicating that the Wasabia japonica leaf extract can effectively reduce the melanin produced by melanocytes due to blue-light irradiation.

### EXAMPLE 6 Cell Experiment—Inhabitation of the Production of Tyrosinase by Wasabia japonica Leaf Extract

2 mL of culture medium (Dulbecco's modified Eagle's medium (DMEM), 1% of penicillin-streptomycin (from Gibco), and 10% of fetal bovine serum (from Gibco)) containing B16F10 cells (ATCC:CRL: 6475) was inoculated in each well of a 6-well plate to allow each well has 1.5×10⁵ cells for culture at 37° C. for 24 h.

Then, the culture medium was removed carefully without disturbing attached cells. 2 mL of fresh DMEM was added to three wells for a control group, and 2 mL of Wasabia japonica leaf extract sample with a concentration of 1 mg/mL was added to the other three wells for an experimental group, to react for 48 h. The Wasabia japonica leaf extract sample was a solution containing 1 mg of diluted Wasabia japonica leaf extracted liquid per mL prepared from the Wasabia japonica leaf extract prepared as in Example 1 and the foregoing diluted Wasabia japonica leaf extracted liquid with a culture medium.

The cells were washed twice with 1× PBS (from Gibco) after the culture medium was removed. Trypsin-EDTA was then added to act on the cells for 3 min, and the suspended cells were collected in a 15 mL centrifuge tube and centrifuged at 400 × g for 5 min to pellet the cells.

Then, after the pelleted cells were washed twice with 1× PBS, the cells were suspended with 200 μL of cell lysis buffer, and the cells were mixed by shaking and then centrifuged at 12,000 × g for 20 min.

The supernatant was removed into a 1.5 mL centrifuge tube to undergo the detection of protein concentration: A. the Bio-rad dye reagent and deionized water were mixed (in a volume ratio of 1:4), and 500 μL of the mixed solution was dispensed into each microcentriftwe tube; B. 10 μL, 8 μL, 6 μL, 4 μL, 2 μL, 1 μL, and 0 μL of BSA with a concentration of 2 mg/mL were respectively added into the microcentrifuge tubes to prepare protein with a standard concentration; and C. 2 μL of test sample was added for detection. 200 μL of the reacted test sample was placed in a 96-well plate, and the absorbance at 595 nm was measured by an ELISA reader (from BioTek).

Including the control group, 400 μg of protein was added into each well, and 90 μL of cell lysis buffer was then added into each well. In a dark environment, 10 μL of 10 M L-Dopa was added at 37° C. for observation every 10 minutes until the suspension turned black. Then, the absorbance at 405 nm was measured.

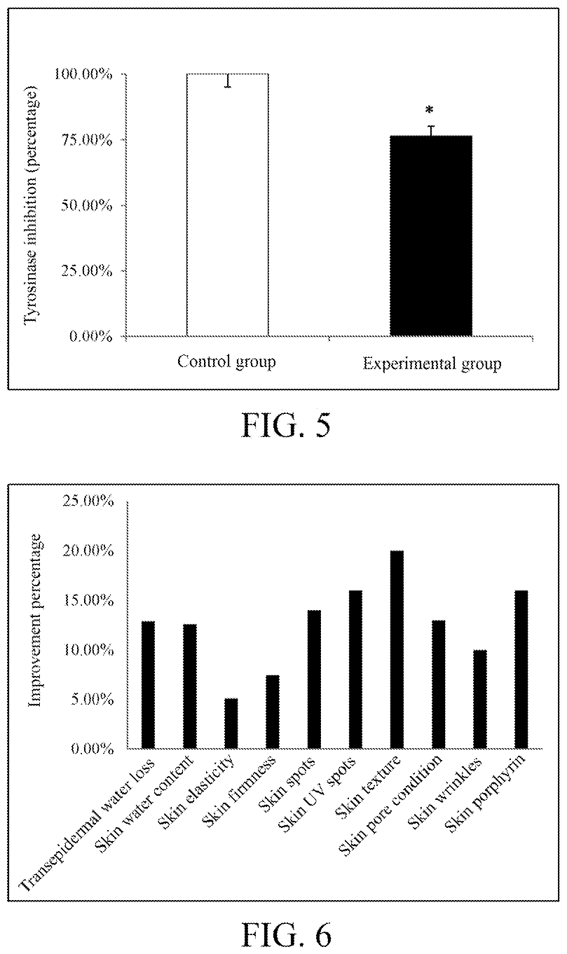

The analytical formula for tyrosinase content was: tyrosinase inhibition (%)=(sample absorbance/control group absorbance×100%). The obtained value was then statistically analyzed using Microsoft EXCEL software by student's t-test. The result was shown in FIG. 5.

It can be learned from the result shown in FIG. 5 that the tyrosinase activity then reduced by about 24% in the case of containing the Wasabia japonica leaf extract of the examples in the present disclosure. Therefore, by using the Wasabi japonica leaf extract of the examples in the present disclosure, the tyrosinase activity can indeed be reduced, and the production of melanin can also be reduced, so that the Wasabia japonica leaf extract can be used in the components of related compositions for reducing skin dark spots and whitening skin.

### EXAMPLE 7: Human Subject Experiment—Internal Use of Wasabia japonica Leaf Extract

Used sample: 50 g/bottle of beverage containing the Wasabia japonica leaf extract of the present disclosure (each 50 g of beverage containing 3 g of the Wasabia japonica leaf extract (that is, 6 wt %), prepared from water and the Wasabia japonica leaf extract). In some other embodiments, each person can have at most 5 g intake of the Wasabia japonica leaf extract per day. The Wasabia japonica leaf extract was prepared by the method as in Example 1 with a Brix degree of 8

Number of Subjects: 10 Subjects Aged from 25 to 40.

Experimental method: each of the subjects drank a bottle of the beverage containing the Wasabia japonica leaf extract of the present disclosure (each bottle contains 3 g of the Wasabia japonica leaf extract) daily for 56 days (i.e., 8 weeks). Before drinking (with the face cleaned, week 0) and 56 days after drinking, values of the facial skin were recorded by using corresponding devices and measurement methods according to different detection items, and photos before and after drinking were taken. (When the detection was carried out before and, after drinking, the temperature and humidity of the detection region where the subjects were located were consistent to reduce the influence of external temperature and humidity on the skin).

Detection items: 1. transepidermal water loss, 2. skin water content, 3. skin elasticity, 4. skin firmness, 5. skin spots, 6 skin spots, 7. skin texture, 8. skin pores, 9, skin wrinkles, and 10. porphyrin (Cutibacterium acnes secretion).

1., Transepidermal Water Loss

The detection was carried out by using a skin water loss detection probe Tewameter® TM 300 (C+K Multi Probe Adapter System, Germany) purchased from Courage+Khazaka electronic, Germany. A cylindrical cavity with open ends is used as the detection probe to form a relatively stable test environment on the skin surface, and the water vapor pressure gradient at two different points is measured to calculate the amount of transepidermal water loss evaporated through the epidermis layer, so as to evaluate the water loss on the skin surface,

2. Skin Water Content

The detection was carried out by using a skin water content detection probe Corneomete® CM825 (C+K Multi Probe Adapter System, Germany) purchased from Courage+Khazaka electronic, Germany. The detection probe is used based on the principle of capacitance for measurement. When the water content changes, the capacitance value of the skin also changes, so that the water content of the skin surface can be analyzed by measuring the capacitance value of the skin.

3. Skin Elasticity and 4. Firmness

The detection was carried out by using a skin elasticity detection probe Cutometer® MPA580 (C+K Multi Probe Adapter System, Germany) purchased from Courage+Khazaka electronic, Germany. The test principle is that, based on the principle of suction and stretching, a negative pressure is formed on the surface of the to-be-tested skin to suck the skin into the detection probe, the depth of the skin sucked into the probe is measured through the optical test system, and the skin elasticity and firmness are then calculated by software analysis.

5. Skin Spots

The detection was carried out by using a VISIA high-end digital skin quality detector purchased from Canfield, USA. High-resolution skin images were taken through visible light, facial skins before and after drinking were compared and analysis was carried out according to the quantity and area of visible pigment spots by using built-in software. The higher the measurement value is, the more visible spots are.

6. Skin UV Spots

The detection was carried out by using a VISIA high-end digital skin quality detector purchased from Canfield, USA. The facial skin was photographed through UV light. Ultraviolet light can be absorbed by melanin to improve the visibility of pigment spots, so as to detect melanin spots on the epidermis layer that are invisible. The higher the measurement value is, the more UV spots are.

7. Skin Texture

The skin texture was detected by using a VISIA high-end digital skin quality detector purchased from Canfield, USA. The principle is that, high-resolution skin images were taken through visible light, and the roughness analysis was carried out according to the unevenness of the skin by using built-in software. The higher the measurement value is, the rougher the skin is.

8. Skin Pore Condition

The detection was carried out by using a VISIA high-end digital skin quality detector purchased from Canfield, USA. The facial skin before and after drinking was photographed through a high-resolution camera lens. Standard white light irradiation causes shadows in the pores, and the pores will be darker than the surrounding skin, so that the pores can be detected, Then, a value is obtained according to the pore quantity and area analysis by using software. The higher the value is, the more the pore quantity and area are.

9. Skin Wrinkles

The detection was carried out by using a VISIA high-end digital skin quality detector purchased from Canfield, USA. The facial skin before and after drinking was photographed through a high-resolution camera lens. The change of the skin shadow is detected by standard white light irradiation to detect the texture position and obtain a value that can represent the smoothness of the skin.

10. Porphyrin (Cutibacterium Acnes Secretion)

Cutibacterium acnes will secrete porphyrin when it grows, and the porphyrin will produce fluorescence when exposed to ultraviolet light, so that the detection can be carried out by using a VISIA high-end digital skin quality detector purchased from Canfield, USA. The facial skin was photographed by using the detector through UV light to detect the content of skin porphyrin. A high measurement value indicates that Cutibacterium acnes grows vigorously and is prone to producing acne.

Refer to FIG. 6. This figure shows the average improvement percentage of the skin condition of the subjects after drinking the beverage containing the Wasabia japonica leaf extract of the present disclosure for 8 weeks, and the improvement percentage accounts for 100% of the total number of subjects. The transepidermal water loss is improved by 12.9%; the skin water content is increased by 12.6%; the skin elasticity is increased by 5.1%; the skin firmness is increased by 7.4%; the skin spots are improved by 14%; the skin UV spots is improved by 16%; the skin texture is improved by 20%; the skin pore condition is improved by 13%; the skin wrinkles is improved by 10%; and the skin porphyrin (Citubacterium acnes secretion) is improved by 16%. It can be learned that the Wasabia japonica leaf extract of the present disclosure has the effects of fading spots, moisturizing, and improving skin fineness, and can help inhibit the production of acne.

Based, on the foregoing, the present disclosure proves that the Wasabia japonica leaf extract can inhibit the production of AGEs and effectively delay skin aging, and the Wasabia japonica leaf extract also can achieve the effects of anti-inflammation and inhibiting the production of acne by inhibiting the gene expression of IL-8 and the secretion of IL-8. Therefore, the Wasabia japonica leaf extract of the present disclosure has the effects of anti-glycation and anti-acne. In addition, the present disclosure also proves that the Wasabia japonica leaf extract can inhibit the production of tyrosinase to achieve the effects of whitening and inhibiting the production of melanin. In terms of the human subject experiments, the present disclosure proves that the Wasabia japonica leaf extract can effectively improve various skin aging conditions, for example, reducing the amount of water lost through skin, increasing skin water content, improving skin elasticity, improving skin firmness, fading skin spots, fading skin UV spots, improving skin texture, improving skin pore condition, reducing skin wrinkles, sand reducing skin porphyrin.

What is claimed is:

1. A method for inhibiting skin acne formation and/or reducing Cutibacterium acnes secretions, comprising administering to a subject in need thereof a composition comprising an effective amount of a Wasabia japonica leaf extract, wherein the Wasabia japonica leaf extract is extracted from a Wasabia japonica leaf with a solvent, and the solvent is water.

2. The method according to claim 2, wherein the Wasabia japonica leaf extract is used for inhibiting skin acne formation by regulating the gene expression of anti-inflammatory genes.

3. The method according to claim 3, wherein the anti-inflammatory gene is an interleukin-8 (IL-8) gene.

4. The method according. to claim 1, wherein the Brix degree of the Wasabia japonica leaf extract is 7.5-8.5.

5. The method according to claim 1, wherein the composition is a cosmeceutical composition, a health food composition, or a cosmetic composition.

6. The method according to claim 1, wherein the liquid-solid ratio of the solvent to the Wasabia japonica leaf is 10:1-20:1.

7. The method according to claim 1, wherein the Wasabia japonica leaf extract is extracted at 70-100° C.

8. A method for reducing, advanced glycation old-products (AGEs), comprising administering to a subject in need thereof a composition comprising an effective amount of a Wasabia japonica leaf extract, wherein the Wasabia japonica leaf extract is extracted from a Wasabiz japonica leaf with a solvent, and the solvent is water.


SEQUENCE LISTING (2 sequences)

SEQ ID NO: 1 - DNA, 22 bp, Artificial Sequence
  IL-8-F
ttttgccaaggagtgctaaaga

SEQ ID NO: 2 - DNA, 21 bp, Artificial Sequence
  IL-8-F
aaccctctgcacccagttttc